(19) United States
(12) Patent Application Publication
(10) Pub. No.: US 2009/0306570 A1
(43) Pub. Date: Dec. 10, 2009

(54) TUBULAR COMPRESSION BANDAGE

(75) Inventor: Hans B. BAUERFEIND, Zeulenroda-Triebes (DE)

Correspondence Address: BIRCH STEWART KOLASCH & BIRCH, PO BOX 747, FALLS CHURCH, VA, 22040-0747, US

(73) Assignee: BAUERFEIND AG, Zeulenroda (DE)

(21) Appl. No.: 12/478,458

(22) Filed: Jun. 4, 2009

(30) Foreign Application Priority Data
Jun. 5, 2008 (DE) 10 2008 026 868.2

(51) Int. Cl. A61F 13/00

(52) U.S. Cl. 602/53

(57) ABSTRACT

The invention relates to a tubular compression bandage made of a textile hosiery which is largely inelastic laterally to the longitudinal direction and is elastic in the longitudinal direction, and which is adapted in tubular form to the contours of the limb in such a way that, when the bandage is applied, it snugly surrounds the limb, the bandage being divided by at least one closure running in the longitudinal direction, such that, when the closure is open, the bandage is applicable to the limb, and when the closure is closed, the bandage firmly surrounds the limb. The bandage material is designed by an elastic, porous knitted fabric having incorporated, circumferential inelastic tension threads for forming a lateral inelasticity, while maintaining the longitudinal elasticity, and the bandage is provided with a window which is held closed or completely or partially open by a textile cover which is made of longitudinally and laterally elastic material and is adapted to the window.

The invention relates to a tubular compression bandage made of a textile hosiery which is largely inelastic laterally to the longitudinal direction and elastic in the longitudinal direction, and which is adapted in tubular form to the contours of the limb in such a way that, when the bandage is applied, it snugly surrounds the limb, the bandage being divided by at least one closure running in the longitudinal direction, such that, when the closure is open, the bandage is applicable to the limb, and when the closure is closed, the bandage firmly surrounds the limb.

A bandage is described in DE 199 46 019 A1, which is composed of an elastic textile base material for the treatment of leg ailments and therefore acts as a compression hose. A rhombic lattice is integrated into the base material of this compression hose, which ensures that, when stretched longitudinally, the compression hose applies a correspondingly higher compression pressure. In bandages or compression hose of this type, the pressure applied to the individual sites on a relevant limb lacks evenness over the relevant limb, e.g. an arm or a leg, depending on the diameter of the individual sites on the relevant limb. For example, the greater diameter in the calf region produces a higher compression pressure than in the region near the ankle joint. Such an uneven distribution of compression pressure along a bandage of this type is often undesirable for reasons of medical treatment.

A bandage of the design mentioned above is known from U.S. Pat. No. 7,073,282 B2. In this bandage, a piece of hosiery material is wrapped around the lower leg, the dimensions thereof being such that the piece of hosiery material may be snugly closed around the lower leg with the aid of a closure (e.g. zipper) running lengthwise along the lower leg. To achieve a desired therapeutic effect on the lower leg, the bandage material is impregnated with latex, and a circumferential, non-extendable thread is coiled and incorporated into the latex coating, this thread being anchored in the dried latex layer and preventing a circumferential elasticity, while maintaining the longitudinal elasticity. The problem of anchoring the circumferential, inelastic thread solved by the latex layer inevitably produces a seal of the bandage, whereby unavoidable sweat on the lower leg is not removable, which means that constant moisture arises beneath the bandage, which is particularly uncomfortable for the wearer and is frequently therapeutically undesirable.

The object of the invention is to provide a tubular bandage according to the design specified above, which does not differ from normal bandages in terms of porosity, i.e. permits continuous permeability, yet nevertheless has the special feature of being elastic and accessible at sites of special medical treatment, while maintaining longitudinal elasticity without any lateral elasticity. The object is achieved by the fact that the bandage material is designed by an elastic, porous knitted fabric having incorporated, circumferential inelastic tension threads for forming a lateral inelasticity, while maintaining the longitudinal elasticity, and by the fact that the bandage is provided with a window which is held closed or completely or partially open by a textile cover which is made of longitudinally and laterally elastic material and is adapted to the window.

The bandage according to the invention is therefore based on a knitted material that is frequently used to advantage for bandages and, as a result of its knitted structure, has a high degree of elasticity, which, however, is completely compensated in the lateral direction by the circumferential inelastic thread that is incorporated during the knitting process, so that a source material is obtained for the bandage according to the invention which is extremely suitable, in particular due to its porosity, for achieving a particular therapeutic effect by the effect of the longitudinal elasticity and prevented lateral elasticity, in that any swellings that occur in the limb surrounded by the bandage are immediately absorbed, while the usually necessary accessibility to a certain location, such as the knee joint, is still made possible by the window provided at this site, and a window covering which has an elastic effect in the longitudinal and lateral directions is ensured thereby, so that, on the one hand, the underlying site on the limb is accessible when the window is opened to a greater or lesser degree, for example to change dressings and the like, and any particular swellings that occur in that site may also have an effect because the covering is elastic in both of its directions, i.e. longitudinally and laterally.

A bandage of this type, which is provided with a window, makes it possible, in particular, to treat, for example, a knee joint on which a knee joint operation was previously performed. The bandage may be placed on the leg by opening the longitudinal closures, the relevant site on the leg remaining accessible through the window opening, so that any type of medical care may be provided at this site.

The orthopedic specialist is thus provided with a bandage for particular treatments of limbs, this bandage being easy to produce because a suitably designed knitted fabric is used and the bandage is able to fully deliver its desired therapeutic effect, while maintaining porosity, over the entire region of the limb, i.e. the arms and legs, outside the window.

The bandage according to the invention fits snugly on a limb over the entire length of the bandage and therefore does not form any particularly tightly confined sites or constrictions, but rather lies snugly against the relevant limb along its length, due to a precise design determined by measuring the relevant limb, and therefore applies a gentle, uniform pressure in the same manner over the entire limb. In order for the bandage to be able to be comfortably attached to the limb, the bandage is divided by a closure running continuously in the longitudinal direction, which, when opened, enables the bandage to be easily applied from the side and, after being closed, the bandage lies snugly against the relevant limb in a manner that always applies the same amount of pressure.

If swellings develop in the relevant limb due to any type of influences, these swellings are absorbed by the radially inelastic bandage and thus prevent individual swellings and corresponding accumulations of blood. The bandage stretches tightly around the relevant area of the relevant limb, as a swelling may occur in this region, which, however, is prevented by the fact that the bandage lies snugly against the limb. As a result, a corresponding counter-pressure occurs in sites that have a tendency to swell, due to the practically nonexistent radial extendibility of the bandage, with the result that the relevant limb maintains its natural contour. The application and removal of the bandage does not cause any problems, since the closure may be completely opened and closed for this purpose.

The cover partly or completely closing the window may determine the remaining window opening by using a separate window closure. By opening the window through a corresponding position of its closure, the underlying site remains accessible as far as necessary for any medical care to be provided at this site.

Exemplary embodiments of the invention are illustrated in the figures, where:

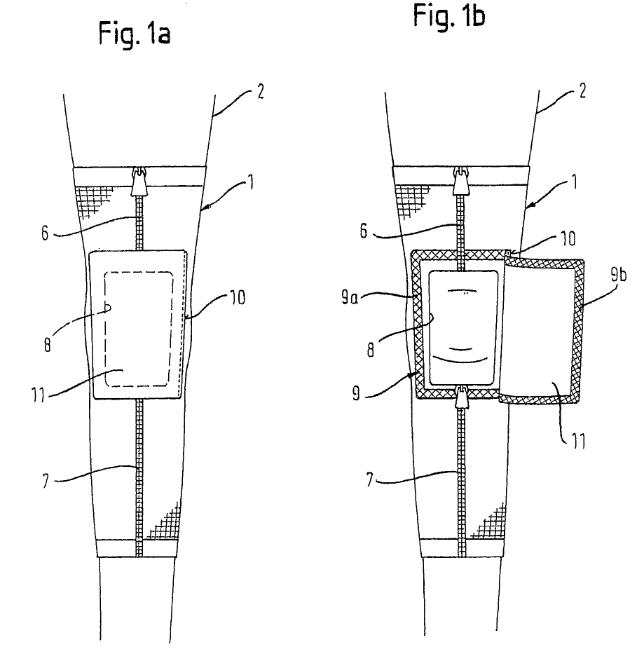

FIGS. 1a and 1b show a bandage for the human leg having a single-panel window in the region of the knee joint (closed in FIG. 1a, open in FIG. 1b), including a closure which is interrupted by the window;

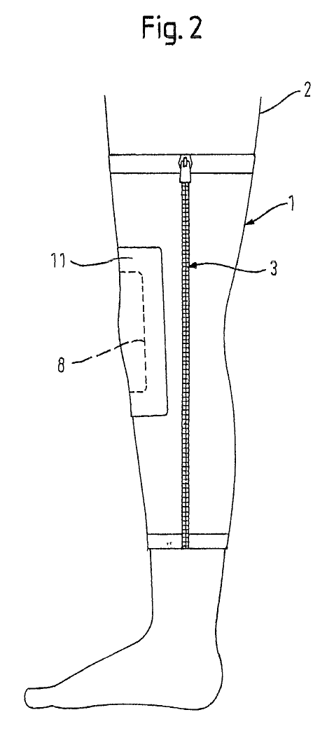

FIG. 2 shows a bandage for the human leg which is similar to the bandage according to FIGS. 1a and 1b, but which has a continuous closure running next to the window;

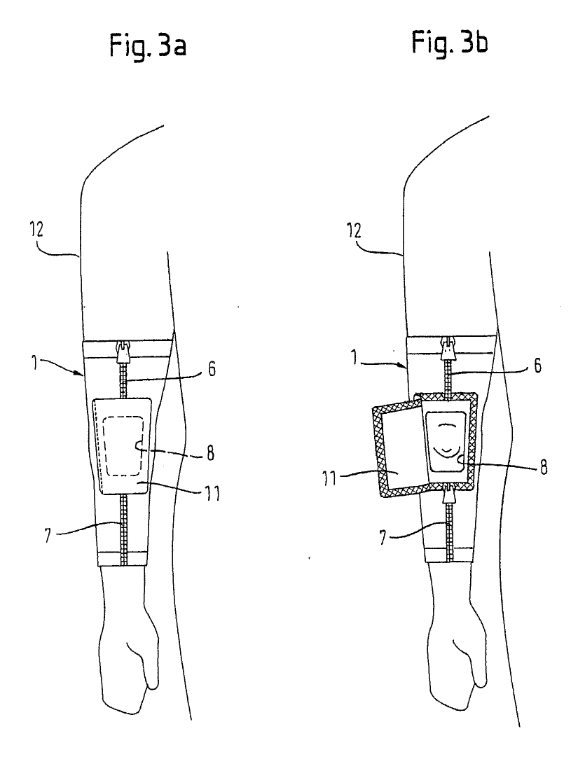

FIGS. 3a and 3b show a bandage for the human arm which has a single-panel window in the region of the elbow joint;

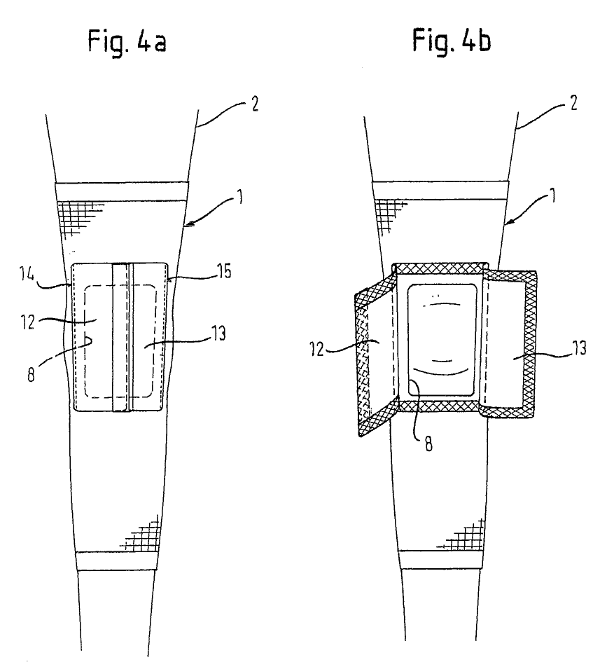

FIGS. 4a and 4b show a bandage for the human leg which is similar to the bandage according to FIGS. 1a and 1b, however having a two-panel window (closed in FIG. 4a, open in FIG. 4b).

Tubular bandage 1 illustrated in FIGS. 1a and 1b is applied to a human leg 2, a zipper 6/7 forming a closure and running along the length of bandage 1 being closed. The foot, which is not illustrated, extends from the lower part of bandage 1. Bandage 1 is made of a knitted fabric containing a closure, which is practically inelastic in the radial direction, due to incorporated, inelastic tension threads surrounding the leg. To cause bandage 1 to lie snugly against leg 2 along the entire length thereof, a measurement of the leg was taken ahead of time in the known manner, as is habitually carried out in orthopedic centers, using known means. According to the data thus obtained, bandage 1 is then individually knitted in such a way that it rests uniformly and snugly against the leg along its entire length when zipper 6/7 is closed.

To apply the bandage, zipper 6/7 is first opened, enabling bandage 1 to surround leg 2 from the side, after which the zipper is closed and the bandage therefore rests snugly against leg 2. The leg is thus uniformly surrounded under even pressure along its entire bandaged length. Any swellings that may arise are unable to have an effect toward the outside, since such swellings are absorbed by the radially inelastic knitted fabric of bandage 1.

Window 8, which is covered by cover 11 (see FIG. 1b), which thus closes window 8 according to the illustration in FIG. 1a, is cut into bandage 1, Cover 11 is joined to the material of bandage 1 on its longitudinal side 10 and thus practically forms a type of hinge in this region, around which cover 11 may be flipped open, as shown in FIG. 1b. The part of leg 1 located beneath window 8 is exposed thereby, in this case, therefore, the knee on which any type of medical care may be provided when window 8 is open. Window 8 may be closed again by flipping back cover 11. In this closed position according to FIG. 1a, cover 11 is held against bandage 1 by Velcro fastener 9, the bandage being provided with Velcro fastening part 9a for this purpose on its remaining sides, the Velcro fastening part being furnished with correspondingly designed Velcro fastener 9b on the edge of cover 11 (see FIG. 1b). When cover 11 is closed, a completely closed bandage is provided, in which only cover 11 has an elasticity for the purpose of protecting the underlying, medically treated sites. Bandage 1 has the two longitudinal zipper parts 6 and 7, which end more or less in the knee region, where window 8 is incorporated into bandage 1. A different fastener may naturally be provided instead of Velcro fastener 9a, in particular a zipper.

FIG. 1 shows a bandage 1, in which closure 3 runs continuously next to window 8.

The bandage according to the invention may also be applied to a different limb, as illustrated in FIGS. 3a and 3b. This limb is a human arm 12, to which bandage 1 is applied and which has components that completely correspond to the components illustrated in FIGS. 1a and 1b. Bandage 1 according to FIGS. 3a and 3b also have a window 8, which is closed in FIG. 3a and open according to FIG. 3b. Closures 6/7 are also designed as illustrated for the bandage according to FIGS. 1a and 1b.

FIGS. 4a and 4b show a modification of the design of a leg bandage according to FIGS. 1a and 1b. Bandage 1 according to FIGS. 4a and 4b includes a window 8 having a two-panel cover. The two panels 12 and 13 of the cover are attached to bandage 1 around the two attachment regions 14 and 15, which act for practical purposes as a hinge. FIG. 4a shows closed window 8 and FIG. 4b shows open window 8. The closure for the bandage is not visible in FIGS. 4a and 4b, but it runs as shown in FIG. 2.

1. A tubular compression bandage (1) made of a textile hosiery which is largely inelastic laterally to the longitudinal direction and is elastic in the longitudinal direction, and which is adapted in tubular form to the contours of the limb in such a way that, when the bandage (1) is applied, it snugly surrounds the limb, the bandage (1) being divided by at least one closure (3; 6, 7) running in the longitudinal direction, such that, when the closure (3; 6, 7) is open, the bandage (1) is applicable to the limb, and when the closure (3; 6, 7) is closed, the bandage firmly surrounds the limb, characterized in that the bandage material is designed by an elastic, porous knitted fabric having incorporated, circumferential inelastic tension threads for forming a lateral inelasticity, while maintaining the longitudinal elasticity, and that the bandage (1) is provided with a window (8) which is held closed or completely or partially open by a textile cover (11, 12, 13) which is made of longitudinally and laterally elastic material and is adapted to the window (8).

2. The bandage according to claim 1, characterized in that the opening of the window (8) is determined by a window closure (9a, 9b) of the cover (11, 12, 13).

3. The bandage according to claim 1, characterized in that the closure is designed as a zipper.

4. The bandage according to claim 1, characterized in that the closure is designed as a Velcro fastener.

5. The bandage according to claim 1, characterized in that the closure is formed by a series of hooks and eyes.

6. The bandage according to claim 2, characterized in that the closure is designed as a zipper.

7. The bandage according to claim 2, characterized in that the closure is designed as a Velcro fastener.

8. The bandage according to claim 2, characterized in that the closure is formed by a series of hooks and eyes.
